(19) United States
(12) Patent Application Publication
(10) Pub. No.: US 2021/0178010 A1
(43) Pub. Date: Jun. 17, 2021

(54) BIOACTIVE WOUND DRESSING

(71) Applicant: COMSATS UNIVERSITY ISLAMABAD, Islamabad (PK)

(72) Inventor: Faiza SHARIF, Lahore (PK)

(73) Assignee: COMSATS UNIVERSITY ISLAMABAD, Islamabad (PK)

(21) Appl. No.: 17/123,625

(22) Filed: Dec. 16, 2020

(30) Foreign Application Priority Data
Dec. 16, 2019 (PK) 842/2019

(51) Int. Cl. A61L 15/28; A61F 13/00

(52) U.S. Cl. CPC A61L 15/28; A61F 2013/00157; A61L 2300/404; A61F 13/00012

(57) ABSTRACT

A cost effective bacterial cellulose wound dressing includes a microbial-derived cellulose loaded with natural antibacterial and regenerative substances. This wound dressing is effective for the treatment of burns and chronic wounds. The dressing keeps the wound moist while absorbing exudates from the wound area. The dressings possess antibacterial qualities to protect the wound from infections and help the area to heal faster through the action of bioactive substances.

### FIELD OF THE INVENTION

The invention relates to a wound dressing comprising a microbial-derived cellulose for treatment of burns and chronic wounds. This invention also relates to a microbial-derived cellulose wound dressing loaded with antimicrobial and regenerative substances that inhibit the wound infection and/or activates the wound healing process. The antimicrobial wound dressing contains antibacterial compounds such as Honey, Royal Jelly (RJ), Nigella sativa extract (NS), wound healing bioactive substances such as RJ and RJ Extract containing a wound healing peptide (Defensin-1) and additionally, Amino Acids L-Arginine and L-Isoleucine as wound healing bioactive compounds.

### DISCUSSION ON THE BACKGROUND

The incidence of burn injuries in the region of South Asia ranges from 112 to 518 per 100,000 cases in a year with a mortality rate of 5.6 per 100,000 cases. There are more cases of female injuries and mortality than males. Being a developing country, the mortality is much higher compared to the 1 death per 100,000 population found in high-income countries. Timely management and wound care can save lives of many of the victims.

Clinical management is needed first, to help reduce pain and protect from environmental factors and then, to heal the burns and wounds. In this regard, wound dressings fabricated from different materials are used to treat burns and abrasions. The dressings range from simple gauze-type dressings to animal derived protein-type dressings such as collagen dressings. In common clinical practice gauze type dressings are sufficient and highly economical for simple abrasions and surgical incisions. However, in cases of chronic wounds, polymer-based dressings are found to be more effective. Advanced polymeric materials with the capability of maintaining moist wound environment have been shown to be more effective than gauze in treating these difficult to heal chronic wounds. Various synthetic and naturally derived polymeric materials have been used to prepare membranes in the treatment of skin disorders.

These materials may be used to fabricate wound dressings with specific properties such as moisture retention and high fluid absorption. Both of these properties, generally not found in gauze-type dressings, promote healing by protecting chronic wounds from infection and maintaining moisture levels in the wound.

Microbial-derived cellulose on the contrary possesses inherent wound healing characteristics eliminating the disadvantages associated with current wound dressings. Microbial-derived cellulose possesses advantages such as nanofibrous, nanoporous structures and unique three-dimensional laminated structures. Microbial cellulose is highly hydrophilic with a water-holding capacity ranging from 60 to 700 times its own weight. Microbial cellulose also demonstrates excellent wet strength and does not break down under compression. Lastly, because of its multi-layered structure, microbial cellulose can be processed to produce a film with novel fluid handling ability. By adjusting the cellulose to liquid ratio, processed microbial cellulose is capable of both donating fluid and absorbing liquid depending on the surface to which it will contact.

Burns and other injuries damage the skin; i.e., a natural barrier between the body and the external environment. Skin prevents transcutaneous water loss; i.e., one of the main causes of mortality due to dehydration. It also protects from harmful environmental UV, chemicals (allergens, irritants) and pathogens. Upon wounding, it is most important that the skin barrier function is restored as quickly as possible. To achieve this, a complex wound healing process involving four overlapping phases namely hemostasis, inflammation, proliferation and tissue remodeling occurs. Similarly, chronic wounds are the wounds which fail to proceed through the normal repair process and manifest an underlying problem like diabetes, venous disease or impaired circulation. Thus, chronic wounds can be broadly categorized as pressure sores, venous and diabetic ulcers depending on the underlying problem. Depending on the cause, various types of wound management treatments and materials are used to address the underlying problem and promote wound healing.

Bacterial cellulose (BC) can be loaded with several antibacterial and wound healing factors. Synthetic antibiotics render bacteria resistant after some time. On the contrary, natural antibacterial materials have been found to have broad spectrum effect. Use of bee products such as honey has been advantageous in healing wounds faster through its regenerative and antibacterial properties. RJ is an acid colloid (3.6-4.2 pH) composed mainly of water, sugar, proteins, lipids, vitamins and some mineral salts. RJ has recently been found to heal wounds faster due to its effect on MMP family of genes which are closely associated with regenerative mechanism. Defensin-1, which is a component of RJ, induces MMP-9 secretion and keratinocyte migration in vitro. In addition, defensin-1 also improves wound closure and re-epithelialization and thus promotes wound healing in vivo. Nigella sativa NS is commonly known as black seed, which has broad spectrum antibacterial properties. Curcumin, a component of Turmeric (Curcuma longa), also plays roles in regeneration and functional reconstruction of skin tissue by stimulating secretion of transforming growth factor β (TGF-β), followed by promoting collagen synthesis and reviving of epithelial cells. Certain non-essential amino acids also play a role in the repair process of wound area. The topical application of L-arginine and L-isoleucine substantially promotes wound healing. However, there is a downside of pure fresh RJ, that is, its short shelf life and temperature dependent fast degradation rate. We isolated the aqueous extract without fat content. It can be stored for longer period of time at 4° C. We combined this extract with honey in a ratio of 1:3, to preserve the proteins and peptides in the extract for a longer period of time.

There are prior examples of inventions related to Bacterial cellulose, for example US Patent 20120129228A to Cornell, where the inventors mention an inexpensive carbon source as nutritional medium that comprises a plant-based seed extract. The seed extract is derived from a plant-based seed comprising soluble sugars. Similarly, another patent WO2005003366A1 to politechnika describes a method for production of bacterial cellulose by using different nutrients for use in wound healing. They do not use any bioactive substance to eradicate infection and do not mention use of any bioactive substance for activated wound healing. U.S. Pat. No. 6,153,413 to Watanabe, et al disclose a method for restoring the various properties of BC even after it is once dried. The patent relates to a method for processing a bacterial cellulose by dehydrating and drying under tension after agitated synthesis of the bacterial cellulose by homogenization. It does not mention any active substances or any activated antimicrobial or wound healing properties.

Use of microbial cellulose in the medical industry has also been explored previously. For example, U.S. Pat. No. 7,704,523 B2 to Serafica et al. disclose the possible use of bacterial cellulose as wound dressings to treat chronic wounds. Serafica et al focus on using statically produced microbial cellulose as wound dressings for treatment of specific types of chronic wounds, including pressure sores, venous and diabetic ulcers. According to their claim, the wound dressing is capable of donating liquid to dry substances and is also capable of absorbing exudating wounds. Serafica et al do not claim any antibacterial or wound healing properties of the bacterial cellulose wound dressings. This patent only declares the use of as synthesized. Another U.S. Pat. No. 6,956,144 B2 to Molan discloses the use of honey as an antibacterial agent for wound healing, but this patent does not use a membrane as a carrier but only increases the viscosity of honey.

The present application involves the use of statically synthesized bacterial cellulose loaded with antibacterial factor and activation factors for increased wound healing. RJ and RJ extract according to our research prove to be more than twice as effective for activating the wound healing process as described in the results and figures in the sections below. This means that the wound can heal more than twice as fast and require much less dressing changes while keeping the wound properly healed with no damage to the newly synthesized collagen and cellular tissue. On the other hand, in current dressing applications, the abrasive damage caused during dressing changes leads to much slower wound healing.

### SUMMARY

Exemplary embodiments provide wet wound dressings based on biosynthesized bacterial cellulose membranes to heal burns and chronic wounds. A series of active substances have been loaded on the membranes to make these dressings antibacterial with a strong, fast regenerative potential. Broad spectrum antibacterial substances such as high quality tested indigenous honey and black seeds (NS/NS) were loaded on the BC membranes to render them impervious to infections. Chronic wounds and burns become untreatable and can be a reason for amputations and mortality that can be avoided by the use of these dressings. Additionally, there are substances which can activate wound healing and regenerative process. Normally tulle gran dressings are used in common clinical practice which contain only some antibiotics and liquid paraffin to avoid sticking on the surface of healing wounds. Frequent dressing changes damage the newly formed regenerated tissue and is painful. These antibacterial regenerative BC wound dressings, in addition to full coverage, have a dual advantage of water retention and nanofibrous, nanoporus structure which does not stick to the surface of the healing wound. The presence of natural healing molecules like defensin-1 containing RJ and RJ extract heals wounds faster. Therefore, less frequent dressing changes are required when using antibacterial regenerative BC dressing, i.e., in place of 7 dressings, only 3 changes are needed. This makes the whole wound management cost effective and comfortable for the patient. In addition, the total cost of the antibacterial regenerative wound dressings is much cheaper and cost effective due to indigenous origin.

The bacterial cellulose is biosynthesized using a very simple and cost-effective technique which does not require any special equipment or use of energy resources. The raw materials are very simple and easily available. The process can be scaled up easily to produce large amounts of BC membranes. The loading of antibacterial and regenerative active substances is also very simple. The membranes can be preserved after sterilization and packaged for a long period of time in wet form and can be made readily available in the time of need.

An exemplary embodiment provides synthesis, purification and loading of bacterial cellulose membranes with honey, RJ, RJ Extract, NS extract, Curcumin, and amino acids. The method includes loading of these on the purified and partially dehydrated membranes using a vacuum pump. The membranes containing loaded bioactives are used as antibacterial and regenerative wound dressings.

The foregoing general description and the following detailed description are exemplary and explanatory and are intended to provide further explanation of the claimed subject matter.

### BRIEF DESCRIPTION OF THE FIGURES

The accompanying drawings, which are included to provide a further understanding of the inventive concept, and are incorporated in and constitute a part of this specification, illustrate exemplary embodiments of the inventive concept, and, together with the description, serve to explain principles of the inventive concept.

FIGS. 1A and 1B illustrates the synthesized and purified BC wound dressings in accordance with an exemplary embodiment.

FIG. 2 Fourier transform infrared spectroscopy (FTIR) spectra of Bacterial cellulose according to an exemplary embodiment.

FIG. 3 Tensile strength measurement of Bacterial Cellulose according to an exemplary embodiment.

FIGS. 4A and 4B Field-emission scanning electron microscopy (FE-SEM) micrograph of dry Bacterial cellulose membrane according to an exemplary embodiment.

FIGS. 5A and 5B A typical antibacterial assay of Bacterial cellulose loaded with honey on S. aureus (5A) and E.coli bacterial strains (5B) according to an exemplary embodiment. BCM (Bacterial cellulose membranes loaded with honey), C (control pure BCM), +ve (honey), Std (standard tetracyclin antiniotic disc).

FIGS. 6A and 6B A typical cell proliferation assay of fibroblasts on Bacterial cellulose RJ Extract (6A) and pure RJ (6B) according to an exemplary embodiment.

FIGS. 7A and 7B A typical Bright field microscopic image of HaKaT (Skin cells) cultured for 3 days on Bacterial cellulose membrane according to an exemplary embodiment (7A) Fluorescent Microscopic Image labelled with DAPI nuclear stain (7B) according to an exemplary embodiment.

FIG. 8 A typical wound healing assay/scratch test of HaKaT cells on BC containing pure RJ 25 ug/ml, 50 ug/ml and 100 ug/ml. BC containing RJ Extract 25 ul, 50 ul and 100 ul according to an exemplary embodiment. Yellow markings show the area free of cells where no cell migration has occurred yet.

FIGS. 9A and 9B A typical quantitative analysis of the cell migration assay/scratch test of HaKaT cells on Bacterial cellulose RJ Extract (9A) and pure RJ (9B) according to an exemplary embodiment.

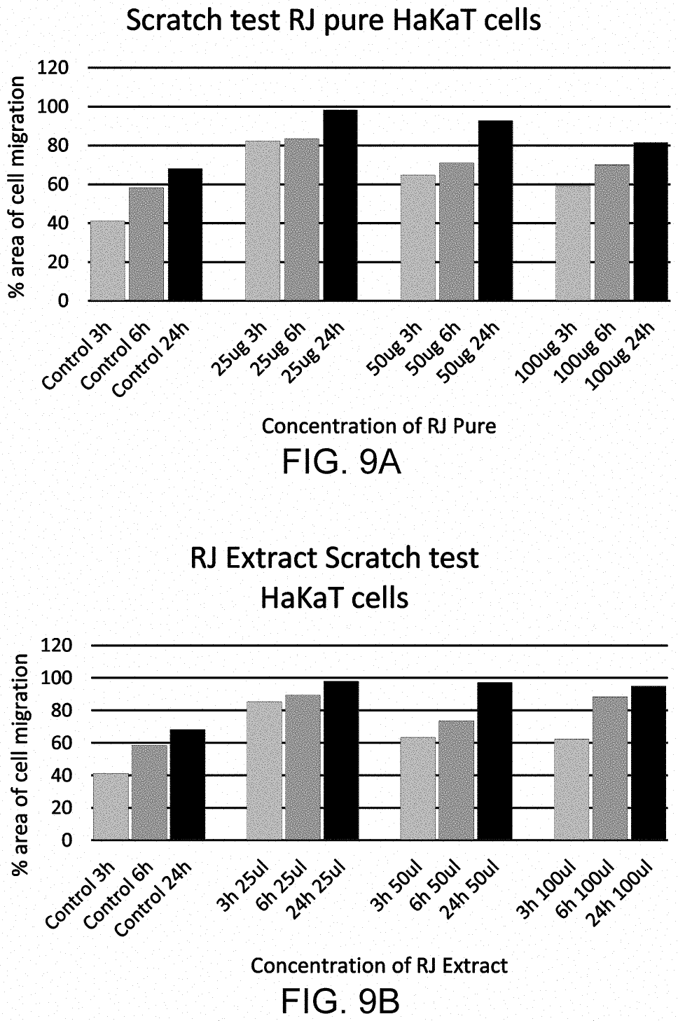

FIG. 10 A flow chart

### DETAILED DESCRIPTION

### Detailed Description of the Illustrated Embodiments

Exemplary embodiments relate to an antibacterial and fast healing wound dressing prepared through biosynthesis. Exemplary embodiments of the invention provide dressings made by bacteria through bacterial synthesis composed of pure cellulose polymer synthesized in the form of membranes. Method of preparation of the sheets employed the use of simple sweetened green tea as media with the provision of low pH through apple cider vinegar, as a result of 2-4-day fermentation, sheets were produced at the air water interface. These membranes were then purified to remove the impurities and bacteria by a simple treatment where sodium hydroxide has been used to remove living bacteria from the membranes to get pure cellulose nanofibrous membranes. These membranes are biomaterials to be used for biomedical applications such as wound dressings.

The BC membranes were impregnated with honey as a broad-spectrum antibacterial agent and RJ extract as a fast healing bioactive. These dressings may be used in various acute and chronic dermal applications. The method is a development of dressings through a process of biosynthesis and use of it as a carrier in which the organic and natural bee products as well a plant extracts were used as wound healing agents, thus enabling a simple, green, and cost effective route for the production of highly efficient wound healing biomedical material. Advantages include room temperatures biosynthesis at 21-30° C., with no need of special instruments or electricity to prepare dressings. Another advantage is that the honey and RJ extract were prepared by centrifugation to isolate the active protein (Defensin-1) which also tested to have twice as fast wound healing potential compared to the current BC membrane dressings loaded with normal saline. Moreover, the method of dressing preparation involves a negligible amount of waste as the inorganic and organic portion of the raw material are almost consumed totally or may be converted into valuable products.

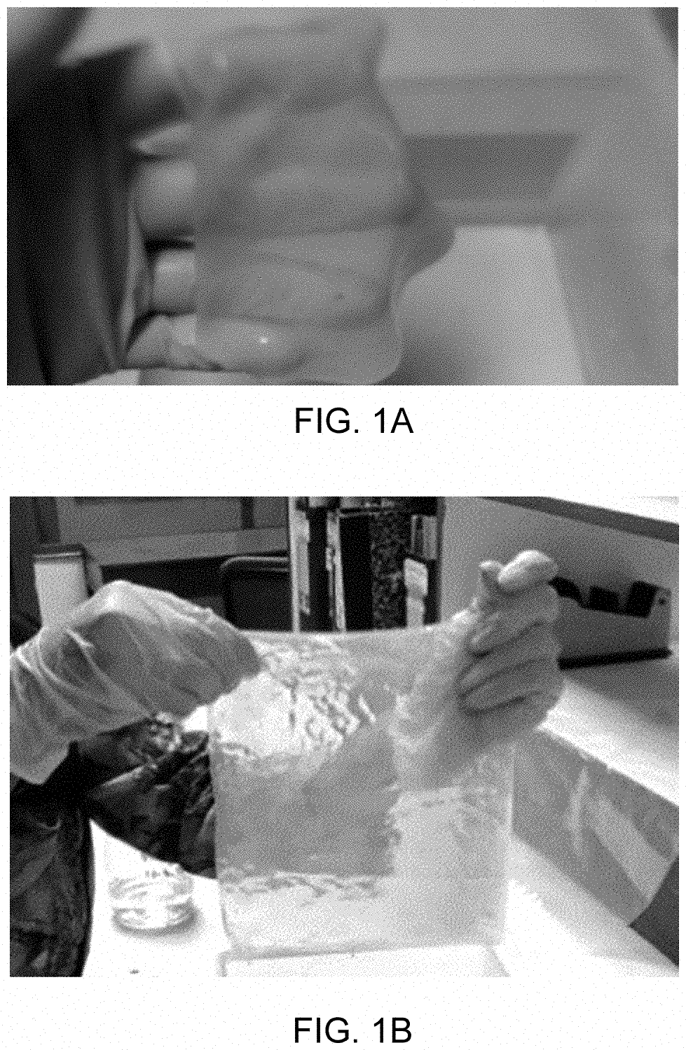

FIG. 1 illustrates an exemplary cellulose membrane synthesized from sweetened green tea. Bacteria isolated from fermentation culture of symbiotic colony of bacteria and yeast were introduced in a culture broth containing sucrose and green tea extract in the presence of apple cider vinegar.

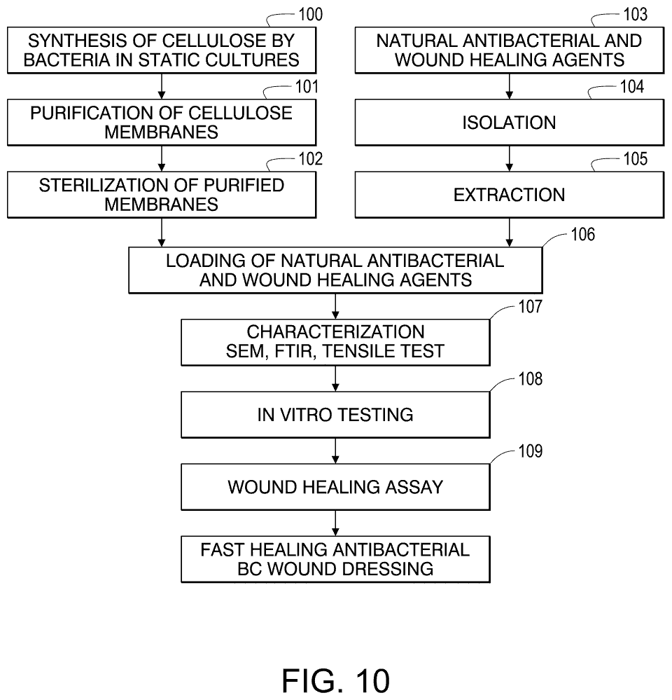

FIG. 10 is a flow chart illustrating aspects of the invention.

In block 100, the method provides preparation of broth. For example, the culture medium can be made from distilled water, to reduce the presence of unnecessary organic matter and other impurities. Green tea 2 gm/L containing antioxidants, polyphenols, flavonoids and vitamins to provide nitrogenous compounds and sugar 100 g/L is added to the culture medium. The bacteria isolated from a symbiotic colony of bacteria and yeast were inoculated at 10% v/v in the culture medium at room temperature 21-26° C. Organic apple cider vinegar was used as acidic content to reduce the pH of media to 3.4-4. The media was left to ferment into beakers at a volume 400 ml/1 L beaker for 2-3 days at static conditions covered with filter paper. The cellulose membranes were formed at the surface of media at air water interface. The resulting membrane ranging in thickness 1.5-5.6 mm is formed on the whole surface area of the beaker. In addition 3 L plastic container/9 cm petri plates can also be used to make variable sized membranes depending upon the time allowed for synthesis.

In block 100, the method provides purification of the synthesized BC membrane by soaking the BC in absolute alcohol for 1-2 min followed by boiling in 0.4% NaOH at 100° C. for 20 min twice.

Then the BC is washed three times with distilled water to remove all impurities. This BC is stored for further use by placing in 30% alcohol at 4° C.

In block 101, the method provides the analysis of BC membrane through FTIR, SEM and Mechanical Testing.

In block 102 the resulting purified cellulose membrane is sterilized for further use by soaking in 70% ethanol and subsequent washing with distilled water.

In blocks 103-105 the extracts of natural antibacterial and wound healing agents were prepared. Fresh high quality honey was sterilized by gamma irradiation and stored till further use in BC membrane at a concentration of 75% of the fluid content. The fresh pure organic RJ extract was prepared by diluting in sterile deionized water at a concentration of 100 mg/ml. The diluted RJ was then centrifuged at a speed of 4000 rpm for 15 min to isolate the protein content. This was named as RJ extract. Pure RJ was diluted to a concentration of 100 ug/ml. Pure concentrated curcumin extract was used at a concentration of 3-4 ug/ml. Arginine and Isoleucine amino acids were used at a concentration of 100 ug/ml.

In block 106 the method provides the loading of natural antibacterial and regenerative agents on the purified BC membranes. The RJ extract was incorporated in combination with honey by placing the BC membrane on the surface of the filter cup and removing 40-50% of water by using vacuum pump. The partially dehydrated membranes were then rehydrated by infusing pure RJ at a conc of 100 ug/ml. In another embodiment the BC was loaded with 100 ug/ml RJ aqueous extract. In another embodiment BC was loaded with honey and RJ aqueous extract at concentration of 750 ug/ml and 100 ug/ml respectively. The active ingredient may comprise 20-30% of honey with NS aqueous extract at a conc. of 100 ug/ml. In yet another embodiment the active ingredient may comprise curcumin or Nigella sativa extract at a conc. of 3-4 ug/ml. In other embodiments, it may be infused with amino acids L-Arginine and L-Isoleucine.

In block 107 the method provides sterilization of fast healing antibacterial BC dressings after packaging by gamma sterilization.

Honey is a natural bee product produced by either Apis dorsata, Apis cerana, or Apis florea, the three indigenous bee species of Pakistan or an imported Apis mallifera. In its concentrated form of invert sugar, glucose, fructose, amino acids, proline, aromatic substances, pigments waxes and pollen grains, unstable compounds such as enzymes, vitamins and sugars. The activity of enzymes, hydroxymethylfurfural (HMF), electrical conductivity makes it an effective wound healing substance. In addition, hyperosmotic nature, low pH and presence of hydrogen peroxide make it a potent antimicrobial component. Acacia honey inhibits growth of bacteria yeast and fungi better than other types of honey as the levels of total sugar, pH, HMF, invert sugar, proline, protein and essential nutrients indicate its high quality. Therefore, this honey is good for use as a topical treatment of antibiotic-resistant pathogenic strains. The quality of most natural raw honey of Pakistan fulfills all the requirements of international standards. Furthermore, the antimicrobial activity of honey against pathogenic bacteria, yeast and fungi confirmed that honey is a broad-spectrum antimicrobial agent and has been used for the healing of various types of wounds since ancient times. The honey used in this case was sourced from Karak area of Pakistan and was then gamma sterilized and kept in sterile tubes till further use.

Examples 1-7 below describe various experiments of synthesis and loading of bacterial cellulose dressings. Example 2 specifically uses fresh RJ loaded onto the purified BC membrane. Example 3 uses RJ aqueous extract as wound healing active. Example 4 uses a combination of RJ extract plus Honey as active wound healing and antibacterial factor. Example 5 uses NS aqueous extract as antibacterial factor loaded on the BC membrane. Example 6 Involves the use of NS and RJ extract as antibacterial and wound healing factor. Example 7 includes the use of amino acids L-Arginine and L-Isoleucine.

### EXAMPLE 1

Example 1 uses the same processes where green tea extract, sugar and apple cider vinegar were used as media in place of Herman Schram media and inoculum of bacteria isolated from 10prior culture was given in a ratio of 10% v/v for synthesis of a Bacterial cellulose membrane instead of a symbiotic colony of bacteria and yeast.

### EXAMPLE 2

Specifically uses fresh RJ loaded onto the purified BC membrane. The purified membranes were partially dehydrated by placing on the filter of a filter cup, then extra water was removed from the membrane using a vacuum pump. While still on the filter surface, the membrane was loaded with fresh RJ diluted in a concentration of 25-100 μg /ml.

### EXAMPLE 3

Uses RJ aqueous extract as wound healing active. The purified membranes were partially dehydrated by placing on the filter of a filter cup, then using the vacuum pump, extra water was removed from the membrane. While still on the filter surface, the membrane was loaded with fresh RJ diluted in a concentration of 25 μl/ml or 50 μl/ml or 100 μl/ml.

### EXAMPLE 4

A combination of RJ extract plus honey was used as active wound healing and antibacterial factor. The purified membranes were partially dehydrated by placing on the filter of a filter cup, then using the vacuum pump, extra water was removed from the membrane. While still on the filter surface, the membrane was loaded with fresh RJ diluted in a concentration of 25 μl/ml or 50 μl/ml or 100 μl/ml added with 75% w/v of pure gamma sterilized honey.

### EXAMPLE 5

NS aqueous extract was used as antibacterial factor loaded on the BC membrane. The purified membranes were partially dehydrated by placing on the filter of a filter cup, then using the vacuum pump, extra water was removed from the membrane. While still on the filter surface, the membrane was loaded with NS aqueous extract (100 g Seeds of NS were weighed and cleaned grinded and added to 1000 ml distilled water in a conical flask for 24 h.) Then the debris was removed by filtration and extracts were used in diluted concentration of 25 μ/ml or 50 μl/ml or 100 μl/ml.

### EXAMPLE 6

Involves the use of Curcumin as antibacterial and wound healing factor. Curcumin extract was used as antibacterial factor loaded on the BC membrane. The purified membranes were partially dehydrated by placing on the filter of a filter cup, then using the vacuum pump, extra water was removed from the membrane. While still on the filter surface, the membrane was loaded with Curcumin aqueous extract (concentration of 3-4 μl/ml). Added to this RJ extract was also loaded in concentrations of 100 μl/ml.

### EXAMPLE 7

Includes the use of amino acids. The purified membranes were partially dehydrated by placing on the filter of a filter cup, then using the vacuum, pump extra water was removed from the membrane. While still on the filter surface, the membrane was loaded with L-Arginine, a non-essential amino acid that plays a key role in protein and amino acid synthesis. It is essential for efficient wound repair, and immune function. L-Arginine and L-Isoleucine is a necessary component of the process of tissue repair as inflammatory cells and fibroblasts use this within the wound for proliferation and as a source of energy, protein and nucleic acid synthesis and collagen deposition.

### Fourier Transform Infrared Spectroscopic Analysis of Bacterial Cellulose

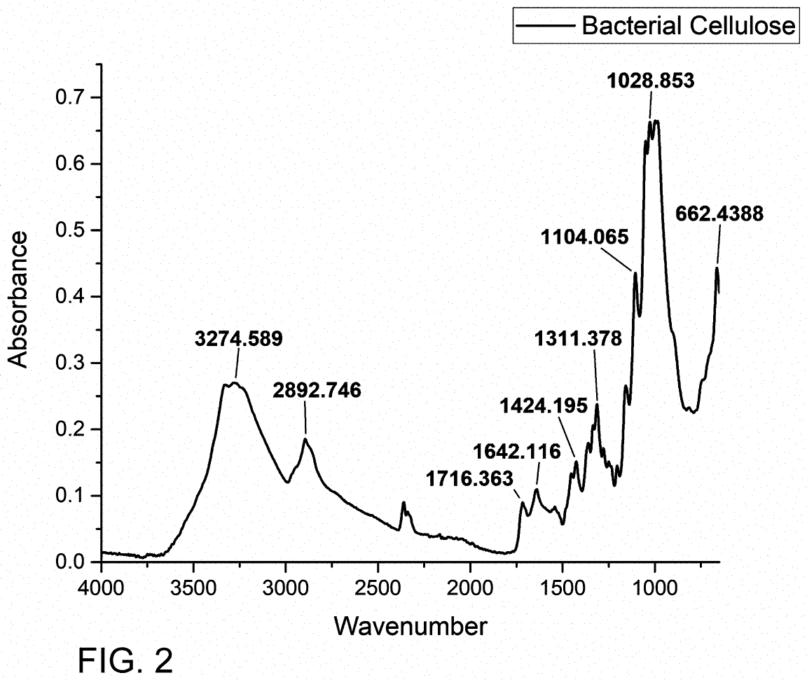

The FTIR spectra were recorded for Bacterial cellulose synthesized in sweetened green tea to identify the characteristics peaks as depicted in FIG. 2. Those assignments agree with the literature. Infrared bands attributed to OH-stretching are located at 3410 cm−1. The majority of the bands corresponding to CH-stretching are located at 2900 cm−1, and the expected absorption bands were in fact observed in the BC spectrum in FIG. 2. H—O—H bending of absorbed water is visible at 1642 cm−1. These data provide evidence of formation of pure bacterial cellulose.

### Mechanical Strength Test

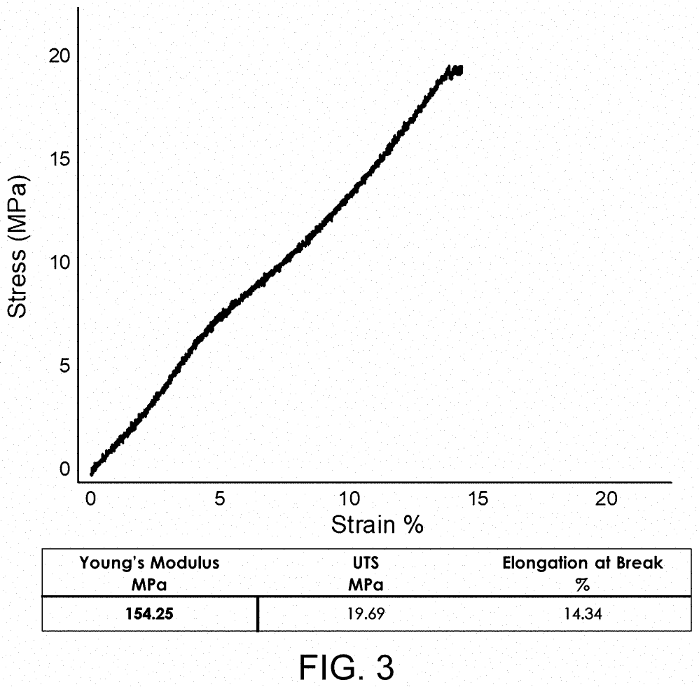

Characterization of the biomechanical properties of the fabricated membranes was investigated by a uniaxial tensile test using a electrodynamic fatigue testing system (Walter+bai ag, LFV-E 1.5 kN, Switzerland). See FIG. 3. To study the ultimate tensile strength, the dried biosynthesized BC membranes were cut keeping uniform width and length which were measured for each sample together with thickness, and then were clamped between two grips of a tensiometer. The length between the clamps was kept uniform for all samples and stress and strain were normalized by area and length of the sample respectively. A load cell of 45 N was used, A ramp test was applied at a rate of 0.1 mm/s and a displacement of 7 mm. Ultimate tensile strength was measured as the plateau at the first failure point. The data were obtained by plotting the max strength achieved before the suture ripped through the membrane as N. Youngs modulus was determined to be 154.2 MPa and ultimate tensile strength was calculated to be 19.69 MPa (FIG. 3). This is considered to be a very high strength for any biomaterial, to render the material easy to handle and apply on the wound area.

### Scanning Electron Microscopic Analysis of Bacterial Cellulose

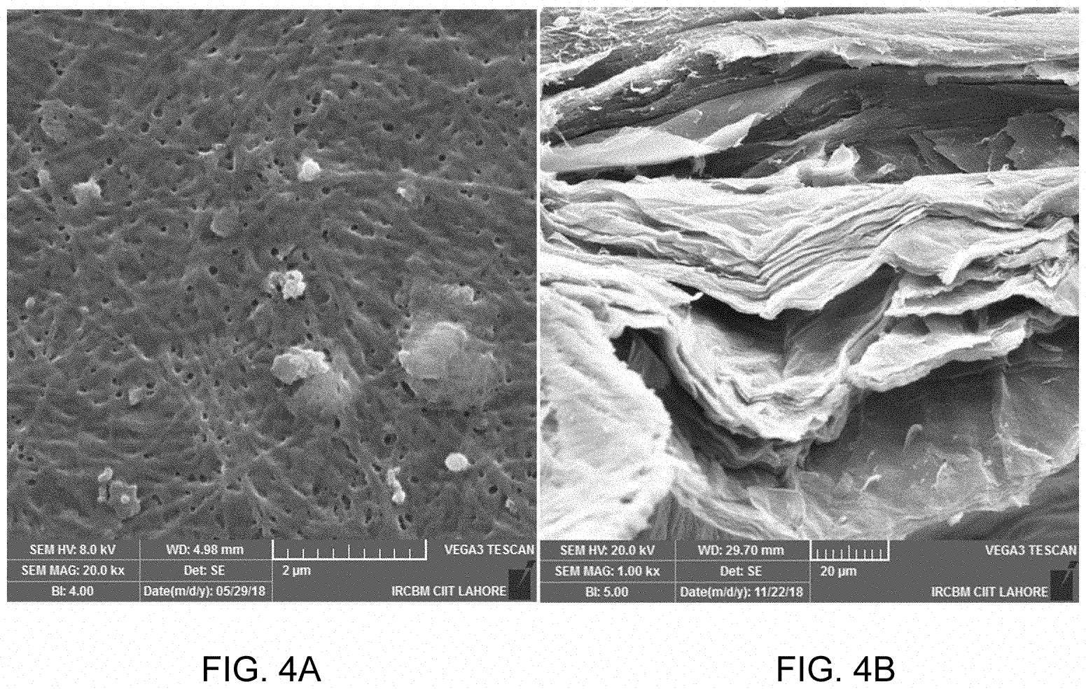

Scanning electron microscopy analysis of the membranes was done to see the morphology of the membranes in dried form. The membranes were nanofibrous and nanoporous as in FIG. 4A. Such nanoporous structure allows molecules and solutions to pass through but do not allow cells to pass. Therefore, these are the best candidates to cover the wounds from outside. The FIG. 4B shows the layered structure of BC. The lamellar sheet like microstructure is responsible for the high mechanical strength and crystallinity of the membranes.

### Antibacterial Assay of Loaded BCM

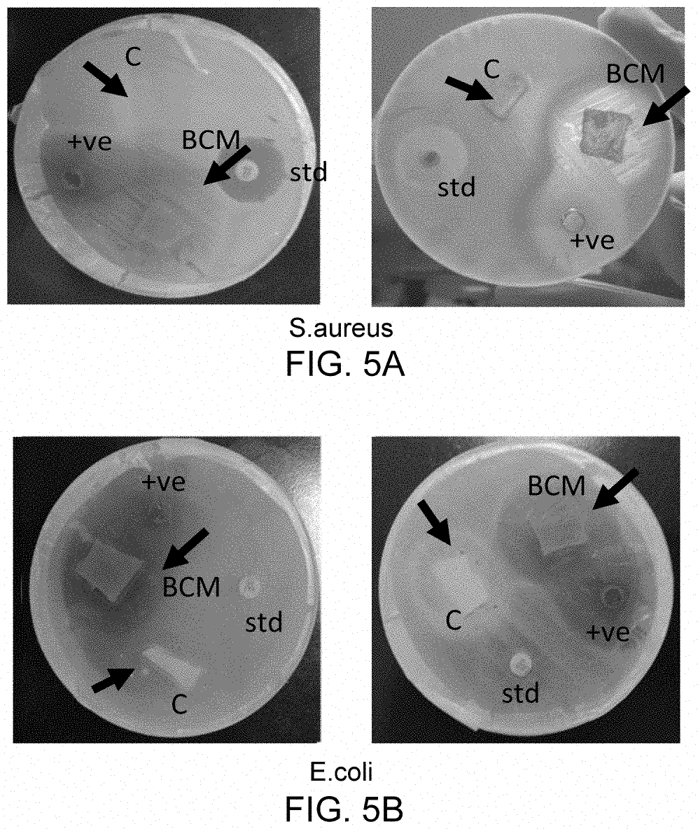

Antibacterial effect of loaded BCM was tested through a zone of Inhibition Test, also called a Kirby-Bauer Test. This is a qualitative method to measure antibiotic resistance and to test the ability of substances to inhibit microbial growth. We tested the effect of honey loaded BCMs on S.aureus and E.coli ATCC strains as shown in FIG. 5. Both species of bacteria were inoculated in the sterile nutrient broth and incubated at 37° C. for 24 h. Petri dishes were prepared by pouring sterilized Mueller Hinton Agar medium and incubating at 37° C. for 24 h. Nutrient broth cultures of S.aureus and E coli bacteria were aseptically swabbed on sterile nutrient agar plates. Wells of 5 mm diameter were made aseptically in the inoculated plates. Standard (Tetracycline, 1 mg/ml) and Control (BCM) were also loaded into the respectively labeled wells. The plates were incubated at 37° C. for 24 h in upright position. The experiment was carried out in triplicate and the zone of inhibition was recorded.

### In Vitro Evaluation

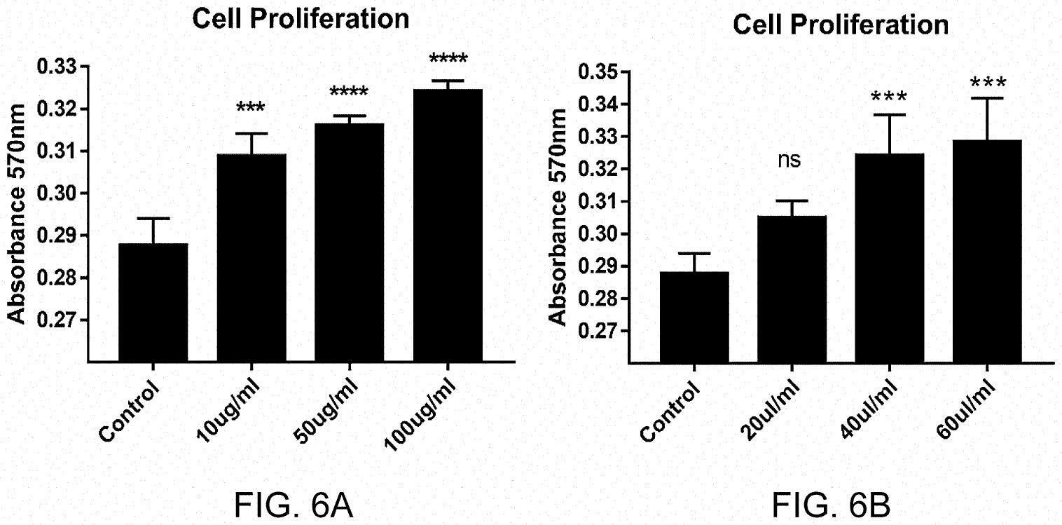

Cell cultures studies were done on the membranes using pre-osteoblast cell lines FIG. 6. In T75 culture flask NIH3T3 cells were expanded in the media that is prepared with a-MEM in which 100 μg/ml of Penicillin/Streptomycin were added and 10% FBS was supplemented, in a humidified incubator at a temperature of 37° C. with 5% CO2 and fresh media changes. After 2 to 3 days the cells were expanded and grown to 90% confluence. Afterwards these cells were detached using Trypsin-EDTA. Before seeding, the cells were counted and 50,000 cells were seeded on each sample in the 24 well plate to check the compatibility of the NIH3T3 cells with BC membranes and membranes loaded with different compounds. Before seeding, the samples were sterilized with 70% ethanol for 2 hours. The samples were washed 3 times with PBS at 15 minutes intervals and the cells were cultured on tissue culture plastic plates without membranes as control.

### Alamar Blue Assay

After 3 days, absorbance measurements of Alamar Blue assay were taken to check the biocompatibility of cells with the BC membranes and the attachment of cells onto the membranes. Based on the metabolic activity of the cells, the Alamar blue has a redox indicator which changes from an oxidized (blue) form to a reduced (red) form as the substrate is taken up by the cells. Cell seeded samples were carefully washed with PBS and 0.5 ml of 1 mM Alamar Blue solution was added. After that, it was incubated for 3 to 4 hours at 37° C. Absorbance plate reader (PR4100 Absorbance Microplate Reader BIO RAD, UK) was used to measure the absorbance at 570 nm. Samples were fixated for DAPI staining and SEM analysis, after the results were taken from the plate reader. The results suggest that there was significantly higher cell proliferation in the cultures containing pure RJ at a concentration of 10 ug-100 ug/ml. This increased with increase in the concentration of RJ as shown in FIG. 6A. Similarly, the BCM containing RJ extract at a concentration of 20 ul/ml-60 ul/ml also show increasing trend in the proliferation of cells with increasing concentration of RJ extract in FIG. 6B. These results suggest that RJ and RJ extract both increase cell proliferation twice as much as without RJ.

### Cell Attachment on BCM Through Fluorescence Microscopy

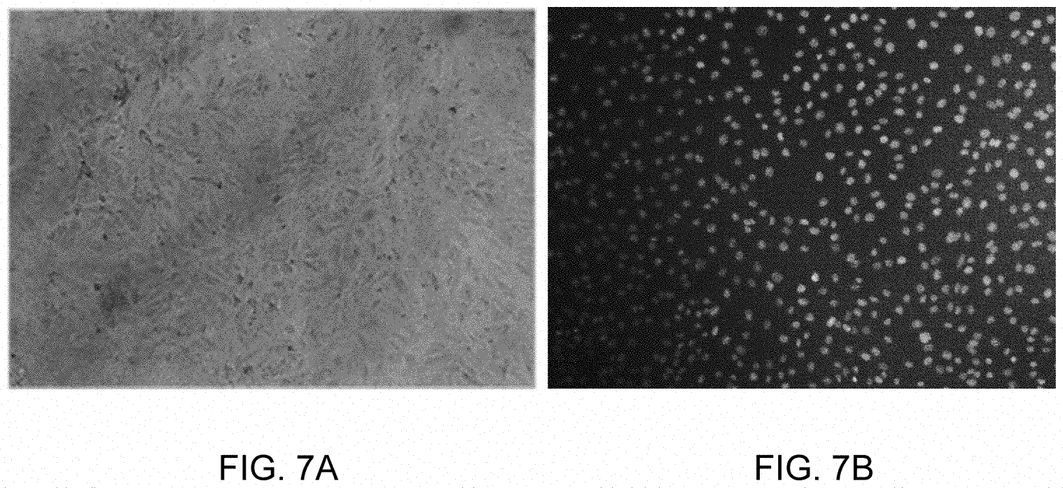

Fluorescence microscopy was done using 89404-464 VWR microscope having 240V with frequency of 50-60 Hz and a halogen lamp of 6V and 30W. The live cells were stained with DAPI and fluorescence images were taken after excitation with UV light with a 460 nm filter at 10×, magnification. The images clearly show cell attachment on membranes. The nuclei stained in blue are clearly seen on the surface of transparent wet BCM in FIGS. 7A and 7B, respectively. Therefore it can be concluded that BCM is a good biomaterial for cell attachment and proliferation, thus possesses regenerative potential.

### Scratch Test (Wound Healing Assay)

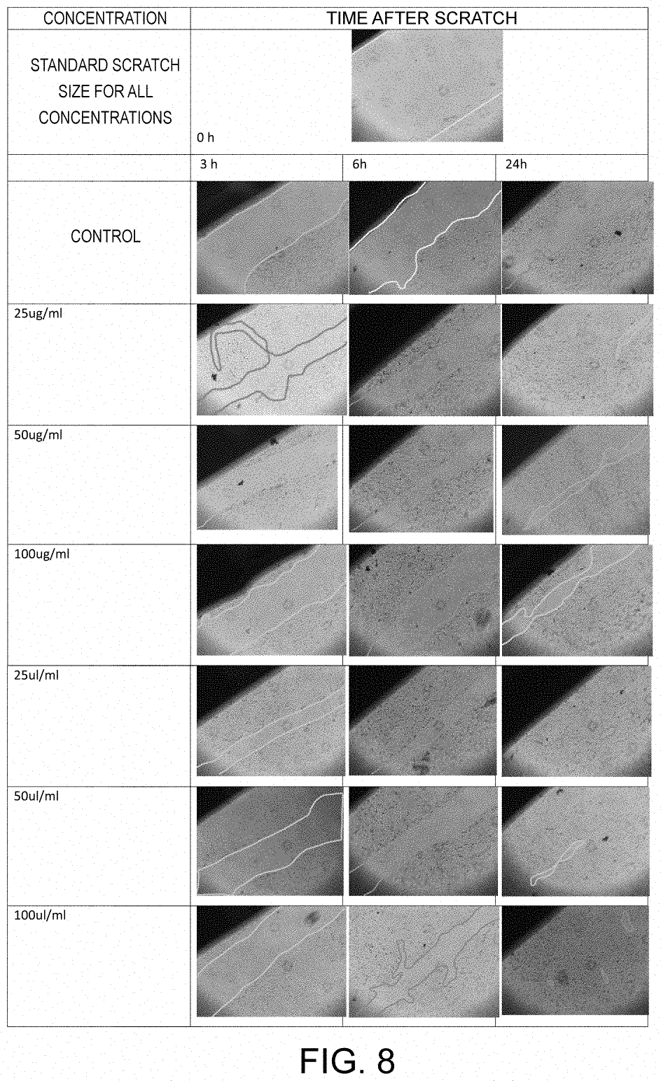

The in vitro scratch assay is an easy, low-cost and well-developed method to measure cell migration in vitro. The skin cells HaKaT were selected as representative for the end user application on skin healing. The cells were seeded on 6 well culture plates to create a confluent monolayer. The dishes were incubated for approximately 6 h at 37° C., allowing cells to adhere and spread on the substrate completely. Then the cell monolayer was scraped in a straight line with a 1000 ul pipette tip. The debris was removed and washed once with DMEM and then DMEM supplemented with 25 50, and 100 ug of RJ and 25 ul, 50 ul and 100 ul of RJ aqueous extract was added. The edges of the scratch were marked with a black marker on the bottom of the dish to be used as reference point for image analysis, and images were then taken under an inverted microscope. The scratched dishes were incubated for 3 h, 6 h and 24 h and imaged again. The images acquired for each sample were further analyzed quantitatively by using computing software Image J. By comparing the images from time 0 to the last time point the distance of each scratch closure was obtained on the basis of the distances that are measured by software. The results, as depicted in FIGS. 8A and 8B, suggest a very fast cell migration in cells containing both pure RJ and RJ extract. This is clearly evident from the images taken after 3-6 and 24 hours post scratch (FIG. 8A). The area covered by the cells to cover the scratch suggests wound healing potential which was measured through image J software and was determined to be significantly higher than a control. There was clear evidence that a concentration of 25 ug/ml was best to activate the cell migration and thus healing compared to 50 and 100 ug/ml.

Similarly as in FIG. 8B, the purified RJ extract also determined a higher cell migration after just 3 h of scratch compared to the other two concentrations.

1. An antibacterial wound healing material, comprising:
cellulose;
a bioactive material; and
an antibacterial agent;
wherein the bioactive material is selected from Defensin-1 (a peptide), Royal Jelly and Royal Jelly extract;
wherein the antibacterial agent is honey.

2. The wound healing material of claim 1, which is in the form of a dressing.

3. The wound healing material of claim 2, wherein the cellulose is bacterial cellulose.

4. The wound healing material of claim 3, wherein the cellulose is in the form of a membrane.

5. The wound healing material of claim 4, wherein the cellulose membrane is incorporated with Royal Jelly or Royal Jelly extract.

6. The wound healing material of claim 4, wherein the cellulose membrane is incorporated with Defensin-1.

7. The wound healing material of claim 1, wherein the Royal Jelly extract is preserved in honey wherein the concentration of RJ extract is 50 ug/ml at a ratio of 1:3 of RJ extract to Honey.

8. The wound healing material of claim 2, wherein the Royal Jelly extract is preserved in honey wherein the concentration of RJ extract is 50 ug/ml at a ratio of 1:3 of RJ extract to Honey.

9. The wound healing material of claim 3, wherein the Royal Jelly extract is preserved in honey wherein the concentration of RJ extract is 50 ug/ml at a ratio of 1:3 of RJ extract to Honey.

10. The wound healing material of claim 4, wherein the Royal Jelly extract is preserved in honey wherein the concentration of RJ extract is 50 ug/ml at a ratio of 1:3 of RJ extract to Honey.

11. The wound healing material of claim 5, wherein the Royal Jelly extract is preserved in honey wherein the concentration of RJ extract is 50 ug/ml at a ratio of 1:3 of RJ extract to Honey.

12. The wound healing material of claim 6, wherein the Royal Jelly extract is preserved in honey wherein the concentration of RJ extract is 50 ug/ml at a ratio of 1:3 of RJ extract to Honey.
